(19) United States
(12) Patent Application Publication
(10) Pub. No.: US 2019/0192115 A1
(43) Pub. Date: Jun. 27, 2019

(54) MEASUREMENT OF BIOMECHANICAL PROPERTIES OF TISSUE

(71) Applicant: THE CLEVELAND CLINIC FOUNDATION, Cleveland, OH (US)

(72) Inventors: Matthew R. Ford, Cleveland, OH (US); William J. Dupps, Cleveland, OH (US)

(21) Appl. No.: 16/098,969

(22) PCT Filed: May 8, 2017
(86) PCT No.: PCT/US2017/031551

§ 371 Date: Nov. 5, 2018

Related U.S. Application Data
(60) Provisional application No. 62/333,234, filed on May 8, 2016.

(51) Int. Cl. A61B 8/10; A61B 8/08; A61B 3/107; A61B 3/00; A61B 3/14

(52) U.S. Cl. CPC A61B 8/10; A61B 8/485; A61B 3/14; A61B 3/107; A61B 3/0025; A61B 8/5215

(57) ABSTRACT

Systems and method are provided for evaluating a biomechanical property of tissue. A shear wave generator induces a shear wave in the tissue. An optical imaging system captures video in the visible light spectrum comprising a series of image frames of the tissue during the shear wave. An image processing component determines, at each of a plurality of locations, a speed at which the shear wave travels within the tissue from the series of image frames. A parameter calculation component calculates a value for the biomechanical property from the determined speed of the shear wave at a plural subset of the plurality of locations.

### RELATED APPLICATION

This application claims priority to U.S. Provisional Patent Application No. 62/333,234, filed May 8, 2016. The entire contents of this application are hereby incorporated by reference.

### TECHNICAL FIELD

This invention relates to imaging systems, and more particularly, to the measurement of biomechanical properties of tissue from an image.

### BACKGROUND OF THE INVENTION

The measurement and understanding of corneal biomechanical properties is an important area of study to improve detection of corneal disease states, and to better understand and alter corneal shape and refraction. Diseases hypothesized to involve a significant disorder of biomechanical strength and drastic alterations to corneal shape include pellucid marginal degeneration, keratoconus, keratoglobus, and post-surgical corneal ectasia. The emergence of corneal collagen crosslinking as a treatment for ectatic corneal disease by stiffening the stroma is a promising treatment, but the mechanical effects have not been completely characterized, largely due to a lack of tools for measuring corneal mechanical properties.

### SUMMARY OF THE INVENTION

In accordance with an aspect of the present invention, a system is provided for evaluating a biomechanical property of tissue. A shear wave generator induces a shear wave in the tissue. An optical imaging system captures video in the visible light spectrum, including a series of image frames, of the tissue during the shear wave. An image processing component determines, at each of a plurality of locations, a speed at which the shear wave travels within the tissue from the series of image frames. A parameter calculation component calculates a value for the biomechanical property from the determined speed of the shear wave at a plural subset of the plurality of locations.

In accordance with another aspect of the present invention, a method is provided for evaluating a biomechanical property of tissue. A shear wave is induced in the tissue. Video in the visible light spectrum, including a series of image frames, of the tissue is captured during the shear wave. At each of a plurality of locations, a speed at which the shear wave travels within the tissue is determined from the series of image frames. A value for the biomechanical property is calculated from the determined speed of the shear wave at a plural subset of the plurality of locations.

In accordance with yet another aspect of the present invention, a system is provided for evaluating a biomechanical property of corneal tissue. A shear wave generator induces a shear wave in the corneal tissue. A camera captures video of a surface of the cornea, including a series of image frames, during the shear wave. An image processing component generates a plurality of difference images between adjacent image frames in the series of image frames, determines the position of the propagating shear wave in each difference image, and determines a speed of the shear wave at each of a plurality of locations from the determined position of the propagating shear wave in the plurality of difference images. A parameter calculation component calculates a value for the biomechanical property from the determined speed of the shear wave at a plural subset of the plurality of locations.

### BRIEF DESCRIPTION OF THE DRAWINGS

The features, objects, and advantages of the hybrid qubit assembly will become more apparent from the detailed description set forth below when taken in conjunction with the drawings, wherein:

FIG. 1 illustrates a system for evaluating a biomechanical property of tissue;

FIG. 2 is a schematic diagram of one example application of a system for evaluating a biomechanical property of tissue;

FIG. 3 is a schematic diagram of another example application of a system for evaluating a biomechanical property of tissue;

FIG. 4 illustrates a method for evaluating a biomechanical property of tissue;

FIG. 5 is a schematic block diagram illustrating an exemplary system of hardware components capable of implementing examples of the systems and methods disclosed herein.

### DETAILED DESCRIPTION

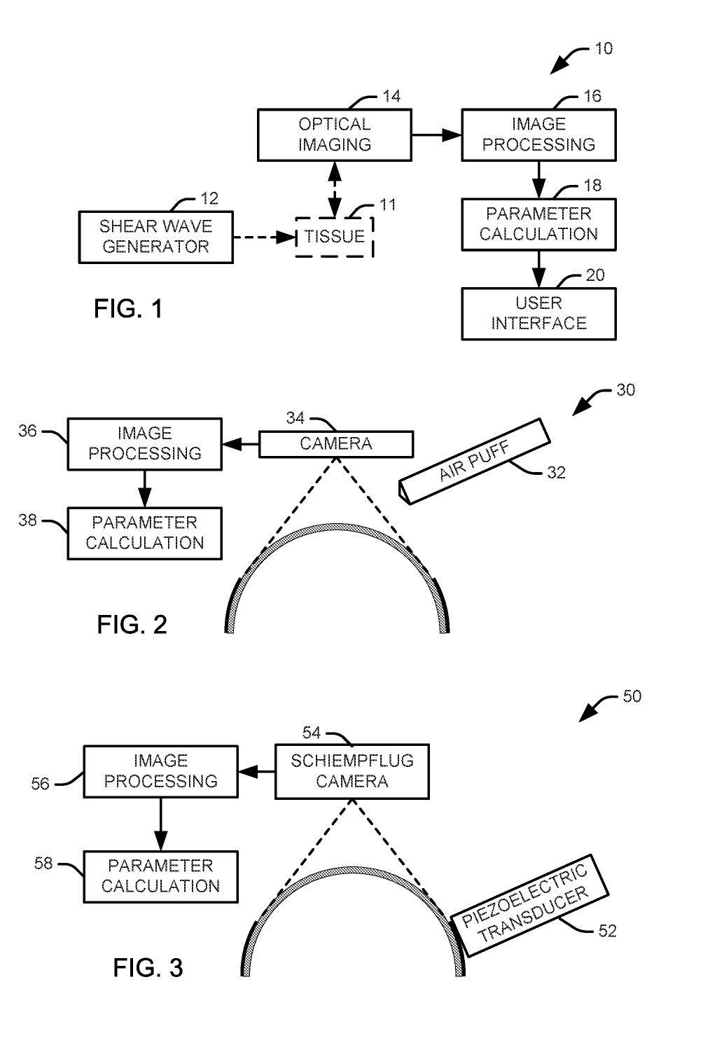

FIG. 1 illustrates a system 10 for evaluating a biomechanical property of tissue. In the illustrated implementation, the system 10 evaluates biomechanical properties of corneal tissue, specifically one or both of the shear modulus and Young's modulus. The illustrated system 10 induces a shear wave within the tissue 11, as a shear wave generator 12, and measures the velocity of the shear wave at locations within the tissue to estimate the material properties of the tissue. It will be appreciated that the shear wave generator can be configured to be placed in physical contact with the tissue 11 to be evaluated, in physical contact with surrounding tissue, or separated from the tissue by a medium that accurately conducts acoustic waves, such as air. In one example, the shear wave generator 12 is implemented as a device for providing an air puff to the eye. It will be appreciated, however, that the shear wave generator 12 can be implemented as any transducer configured to produce mechanical waves within the tissue 11, including ultrasound transducers or a set of piezo-electric bimorph and piezo electric stacks driven by an appropriate control system.

An optical imaging system 14 captures video, comprising a series of image frames, of the tissue 11 during the inducement of the shear wave. In one implementation, the optical imaging system 14 is a camera that images the surface of the tissue as the shear wave propagates. In another implementation, the optical imaging system 14 is an optical section imaging system, such as a Schiempflug camera arrangement, images a cross-section of the tissue that is substantially perpendicular to a surface of the tissue. It will be appreciated that both of these modes can be used in concert to provide a three-dimensional mapping of the movement of the wave through the tissue. The captured video is then provided to an image processing component 16 configured to determine a speed of the shear wave in the tissue 11 from the video at each of a plurality of locations. It will be appreciated that the image processing component 16 can be part of the software, firmware, or circuitry associated with the optical imaging system 14, a completely standalone component comprising either or both of dedicated hardware and software or firmware executed by an associated processor, or distributed across the optical imaging system 14 and a standalone component.

In one example, the image processing component 16 computes difference images between adjacent image frames in the series of image frames, and determines the position of the propagating shear wave in each difference image. Utilizing the difference images, along with knowledge of the spatial scale of the images and the time signature of each image, the local shear wave speed can be calculated at each frame, and a corresponding map of the speed values can be created. A parameter calculation component 18 calculates a value for the biomechanical property for some or all of the plurality of locations within the tissue 11 from the determined speed of the shear wave at the evaluated locations. It will be appreciated that the parameter calculation component 18 can be part of the software, firmware, or circuitry associated with the optical imaging system 14, a completely standalone component comprising either or both of dedicated hardware and software or firmware executed by an associated processor, or distributed across the optical imaging system 14 and a standalone component.

In one implementation, the calculated value is a shear modulus, which can be determined from the calculated speed at each locations as:

G=vₛ²ρ Eq. 1

where vₛ is the measured shear speed and ρ is a density of the tissue. In one implementation, the density of the tissue can be assumed to be substantially constant and can be estimated according to the tissue type.

The calculated values can be provided to a user at a user interface 20 or provided to a modelling component (not shown) for use in modelling the imaging tissue. Such a modelling component can be found, for example, in U.S. Pat. No. 8,346,518, which is hereby incorporated by reference. The illustrated system would provide the biomechanical parameters utilized by this system to provide a patient specific model of corneal tissue.

The inventors have determined that visible light imaging provides an efficient method for measuring shear wave velocity in tissue. Using ultrasound methods would require two cross propagating shear waves, which is slow, relative to ophthalmic imaging, and has poor resolution due to the frequency limits of the ultrasound device. Optical coherence tomography (OCT) would require long scan times or enormous data acquisitions to capture propagating shear wave behavior in more than a single tissue meridian or multiple perturbations to construct a spatial map of properties. This illustrated system 10 can be very inexpensive, using a camera, a relatively low power processing device, and an air puff device or other simple perturbation method. Given the teachings of the present invention, the system 10 could be easily integrated into existing corneal measurement devices with only an increase the specifications of their existing cameras.

FIG. 2 is a schematic diagram of one example application of a system 30 for evaluating a biomechanical property of tissue. In the illustrated implementation, the system 30 evaluates biomechanical properties of corneal tissue, specifically one or both of the shear modulus and Young's modulus, by determining a shear wave speed within the tissue. It will be appreciated, however that the system 30 could also be used to characterize the sclera and the limbal region connecting the cornea and sclera, as well as other ocular structures that can be imaged and that have the potential to propagate a shear wave, such as the crystalline lens, retina, vitreous, and choroid. In other implementations, the system 30 could also be applied to skin or any tissue type that can be physically accessed.

The system 30 includes an air puff generator 32, for directing a puff of air onto the cornea, and a camera 34 positioned to image a surface of an eye of a patient. The air puff generator 32 is positioned near the eye and configured direct air at the cornea as to induce a shear wave within the eye tissue at one or more locations within the eye. This generates a shear wave traveling outward in the cornea from the point of application. The wave speed is in the several to tens of meters per second. and thus will propagate throughout the cornea faster than the patient will react to the air puff. However, the speed is low enough to be captured even at slower imaging frame rates (e.g., 240 fps), and higher frame rates allow higher temporal and spatial resolution. Factors such as pixel count, magnification, and frame rate all influence the effectiveness of the measurement. Typical air puff diameters limit the measurable area of the cornea since a shear wave will not be seen within the impact zone. To allow more complete coverage of the cornea, a smaller air puff or n additional decentered impact(s) can be utilized to provide shear wave data for previously obscured areas.

The camera 34 can be oriented relative to the eye to be co-axial, paraxial or in any configuration that provides an en face view of the tissue surface. Such a view provides a surface view that allows calculation of local wave speed for each corneal radian, that is, along every possible radial angle in a 360-degree reference system) from a single perturbation so that regional differences in superior, inferior, nasal, temporal and oblique properties can be measured as a function of radial orientation and distance from the corneal center or center of the air puff. There are known anatomic and biomechanical property differences that manifest along these orientations owing to the preferred collagen orientations of the cornea. The presence of such mechanical property differences is referred to as anisotropy, and the ability to differentiate the properties along these key orientations in a patient or sample specific manner provides a major advantage over current approaches that lack such spatial and directional resolution.

The captured images can be provided to an image processing component 36 to determine the speed at which the shear wave propagates. In the illustrated implementation, the wave speed propagation can be imaged by subtracting corresponding pixel values from adjacent frames. This procedure is computationally efficient enough to be run within milliseconds. Utilizing the difference maps and knowledge of the spatial scale of the images and the time signature of each image, the local shear wave speed can be calculated at each frame and assigned to the appropriate locations for each frame. It will be appreciated that this calculation can include compensation for the shape of the cornea, which is largely lost in an en face view

From the calculated speed values across frames, either or both of the shear modulus and Young's modulus can be calculated for each location in the cornea and a corresponding map of the values can be created at a parameter calculation component 38. These maps can be superimposed on or compared to corneal topographic maps using identical spatial scales to relate mechanical features to corneal curvature or elevation features, with color scales to represent elastic or shear modulus values as a function of space, and values can be organized into summary variables by region of interest, orientation, or any other parameter useful for clinical interpretation. For a homogeneous isotropic material, Young's modulus can be determined to be approximately three times the shear modulus, as calculated in Eq. 1 above. It is not always appropriate to make the assumption of a homogeneous isotropic material, especially in the cornea. However, given the spatial extent of the acoustic wave at low frequencies, such as frequencies below two kilohertz, this is still a useful simplifying assumption. This calculation may require modification for higher frequency waves with corresponding smaller spatial extent.

One aspect of the invention is the use of the shape of the propagating wavefront from the impulse center as an indicator and measure of regional and directional property differences. The propagating wavefront takes on a shape that would reflect spatial differences in the local mechanical properties of the tissue. For example, in an isotropic material, with regionally and directionally identical mechanical properties, the wavefront would propagate at the same speed from the impulse center as an expanding circle. Anisotropy would produce an irregular wavefront, and that shape, expressed as an absolute shape or its deviation from a circle, can be quantified as another local or cumulative measure of regional and directional material properties and associated diseases or risk states.

FIG. 3 is a schematic diagram of another example application of a system 50 for evaluating a biomechanical property of tissue. In the illustrated implementation, the system 50 evaluates biomechanical properties of corneal tissue, specifically one or both of the shear modulus and Young's modulus, by determining a shear wave speed within the tissue. It will be appreciated, however that the system 50 could also be used to characterize the sclera and the limbal region connecting the cornea and sclera, as well as other ocular structures that can be imaged and that have the potential to propagate a shear wave, such as the crystalline lens, retina, vitreous, and choroid. In other implementations, the system 50 could also be applied to skin or any tissue type that can be physically accessed.

The system 50 includes a piezoelectric transducer 52 and Schiempflug camera arrangement 54. The piezoelectric transducer 52 is positioned on or near the eye and configured to induce a shear wave within the eye tissue. In the illustrated implementation, the piezoelectric transducer 52 includes a piezoelectric bimorph and piezoelectric stacks driven by an arbitrary function generator and a one hundred and fifty volt amplifier. In the illustrated implementation, the piezoelectric transducer 52 is configured to provide a surface deformation of less than four micrometers to ensure compliance with FDA safety protocols. It will be appreciated, however, that amplitudes significantly less than four micrometers can be successfully used in measuring biomechanical properties.

The captured images can be provided to an image processing component 56 to determine the speed at which the shear wave propagates through the cross-section of tissue. In the illustrated implementation, the wave speed propagation can be imaged by subtracting corresponding pixel values from adjacent frames of video. Utilizing the difference maps and knowledge of the spatial scale of the images and the time signature of each image, the local shear wave speed can be calculated at each frame and assigned to the appropriate locations for each frame, with biomechanical parameters calculated from the velocity values as described previously.

The Schiempflug camera arrangement 54 allows visualization of the shear wave in tissue cross-sections as a function of time. Rather than providing en face information on directional wave speed behavior, this system 50 provides depth-dependent sensitivity for quantifying shear wave speed as a function of tissue depth. Much like the surface wavefront shape, the cross-sectional wavefront shape can be used as a measure of depth-dependent elastic and shear moduli for the imaged section. It is known that the cornea has a gradient of mechanical properties from the anterior to the posterior surface, and the ability to measure patient and sample-specific variations in this depth-dependence can be used for evaluating corneal health. For example, a tissue with uniform material properties through the depth dimension would produce a linear shear wave front. A tissue like the cornea with stereotypically greater elastic properties in the anterior portions would produce a wavefront with a slope or nonlinear shape due to faster speeds in the anterior tissue. This shape can be used to fit a function to the depth dependent properties, and this function can be use directly in diagnostic algorithms or for specifying the material properties in computational models. Geometric corrections for viewing angle and corneal curvature can be implemented as needed to correct for the dependence of visualized shear wave speed on these variables.

In one implementation, the en face imaging of FIG. 2 and the cross-sectional imaging of FIG. 3 can be combined, serially or simultaneously, fully characterize the elastic or shear properties of the target tissue. This map can then be used for characterization and diagnosis of corneal biomechanical pathology such as keratoconus, pellucid marginal degeneration, post-operative corneal ectasia and other corneal ectasias, or as an input to finite element analysis or statistical methods to predict structural responses to possible interventions and aid design of surgical parameters to enhance outcome accuracy and reduce surgical risk.

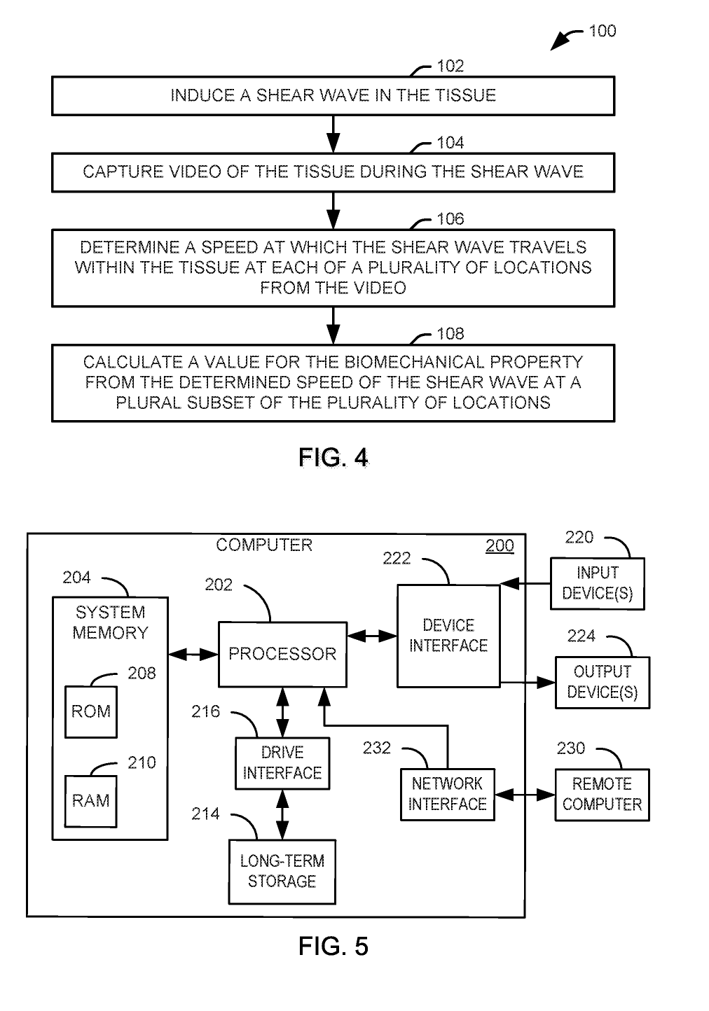

In view of the foregoing structural and functional features described above, a method in accordance with an aspect of the present invention will be better appreciated with reference to FIG. 4. While, for purposes of simplicity of explanation, the method of FIG. 4 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect the present invention.

FIG. 4 illustrates a method 100 for evaluating a biomechanical property of tissue. At 102, a shear wave is induced in the tissue. For example, this can be done via providing an air puff to the tissue, mechanically shaking the tissue, for example, via a piezoelectric transducer, or applying ultrasound to the tissue. At 104, video is captured in the visible light spectrum comprising a series of image frames of the tissue during the shear wave. This can include either or both of capturing video of a cross-section of tissue substantially perpendicular to a surface of the tissue at a Schiempflug camera arrangement and capturing video of a surface of the tissue at a camera.

At 106, a speed at which the shear wave travels within the tissue is determined, at each of a plurality of locations, from the series of image frames. In one implementation, determining the velocity includes generating a plurality of difference images between adjacent image frames in the series of image frames, determining the position of the propagating shear wave in each difference image, and determining a speed of the shear wave at each of a plurality of locations from the determined position of the propagating shear wave in the plurality of difference images. At 108, a value for the biomechanical property is calculated from the determined speed of the shear wave at a plural subset of the plurality of locations. This value can include, for example, either or both of Young's modulus and a shear modulus.

FIG. 5 is a schematic block diagram illustrating an exemplary system 200 of hardware components capable of implementing examples of the systems and methods disclosed herein, such as the imaging and biomechanical analysis system described previously. The system 200 can include various systems and subsystems. The system 200 can be a personal computer, a laptop computer, a workstation, a computer system, an appliance, an application-specific integrated circuit (ASIC), a server, a server blade center, a server farm, etc.

The system 200 can includes a system bus 202, a processing unit 204, a system memory 206, memory devices 208 and 210, a communication interface 212 (e.g., a network interface), a communication link 214, a display 216 (e.g., a video screen), and an input device 218 (e.g., a keyboard, touch screen, and/or a mouse). The system bus 202 can be in communication with the processing unit 204 and the system memory 206. The additional memory devices 208 and 210, such as a hard disk drive, server, stand alone database, or other non-volatile memory, can also be in communication with the system bus 202. The system bus 202 interconnects the processing unit 204, the memory devices 206-210, the communication interface 212, the display 216, and the input device 218. In some examples, the system bus 202 also interconnects an additional port (not shown), such as a universal serial bus (USB) port.

The processing unit 204 can be a computing device and can include an application-specific integrated circuit (ASIC). The processing unit 204 executes a set of instructions to implement the operations of examples disclosed herein. The processing unit can include a processing core.

The additional memory devices 206, 208 and 210 can store data, programs, instructions, database queries in text or compiled form, and any other information that can be needed to operate a computer. The memories 206, 208 and 210 can be implemented as computer-readable media (integrated or removable) such as a memory card, disk drive, compact disk (CD), or server accessible over a network. In certain examples, the memories 206, 208 and 210 can comprise text, images, video, and/or audio, portions of which can be available in formats comprehensible to human beings.

Additionally or alternatively, the system 200 can access an external data source or query source through the communication interface 212, which can communicate with the system bus 202 and the communication link 214.

In operation, the system 200 can be used to implement one or more parts of an imaging system in accordance with the present invention. Computer executable logic for implementing the composite applications testing system resides on one or more of the system memory 206, and the memory devices 208, 210 in accordance with certain examples. The processing unit 204 executes one or more computer executable instructions originating from the system memory 206 and the memory devices 208 and 210. The term “computer readable medium” as used herein refers to a medium that participates in providing instructions to the processing unit 204 for execution, and can include a single medium or multiple, operatively-connected media operating in concert.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications, and variations that fall within the scope of the appended claims.

What is claimed is:

1. A system for evaluating a biomechanical property of tissue, comprising:
a shear wave generator that induces a shear wave in the tissue;
an optical imaging system that captures video in the visible light spectrum comprising a series of image frames of the tissue during the shear wave;
an image processing component that determines, at each of a plurality of locations, a speed at which the shear wave travels within the tissue from the series of image frames; and
a parameter calculation component that calculates a value for the biomechanical property from the determined speed of the shear wave at a plural subset of the plurality of locations.

2. The system of claim 1, wherein the image processing component computes difference images between adjacent image frames in the series of image frames, and determines the position of the propagating shear wave in each difference image.

3. The system of claim 1, wherein the optical imaging system is a camera that captures video of a surface of the tissue.

4. The system of claim 3, wherein the image processing component corrects the determined speed at the plurality of locations for a curvature of the surface of the tissue.

5. The system of claim 3, wherein the image processing component registers the determined velocities at the plurality of locations with an anatomical map of the tissue.

6. The system of claim 3, wherein the image processing component generates, from the video, a shape of the shear wave on the surface of the tissue at a given time after the shear wave is induced, and the parameter calculation component calculates at least one value representing the anisotropy of the tissue from the generated shape.

7. The system of claim 1, wherein the optical imaging system employs optical section imaging to capture video of a cross-section of tissue substantially perpendicular to a surface of the tissue.

8. The system of claim 7, wherein the image processing component generates, from the video, a depth-dependent shape of the shear wave at a given time after the shear wave is induced, and the parameter calculation component calculates at least one value representing one of a depth-dependent elastic modulus and a depth-dependent shear modulus from the generated shape.

9. The system of claim 1, wherein the shear wave generator is a piezoelectric transducer that directly engages the tissue to induce the shear wave.

10. The system of claim 1, wherein the shear wave generator is an ultrasound transducer.

11. The system of claim 1, wherein the shear wave generator delivers an air puff to the eye.

12. A method for evaluating a biomechanical property of tissue comprising:
inducing a shear wave in the tissue;
capturing video in the visible light spectrum comprising a series of image frames of the tissue during the shear wave;
determining, at each of a plurality of locations, a speed at which the shear wave travels within the tissue from the series of image frames;
calculating a value for the biomechanical property from the determined speed of the shear wave at a plural subset of the plurality of locations.

13. The method of claim 12, wherein determining a speed at which the shear wave travels in the tissue comprises:
generating a plurality of difference images between adjacent image frames in the series of image frames;
determining the position of the propagating shear wave in each difference image; and
determining a speed of the shear wave at each of a plurality of locations from the determined position of the propagating shear wave in the plurality of difference images.

14. The method of claim 13, wherein capturing video of the tissue comprises capturing video of a cross-section of tissue substantially perpendicular to a surface of the tissue at a Schiempflug camera arrangement.

15. The method of claim 14, wherein capturing video of the tissue further comprises capturing video of a surface of the tissue at a camera.

16. A system for evaluating a biomechanical property of corneal tissue, comprising:
a shear wave generator that induces a shear wave in the corneal tissue;
a camera that captures video of a surface of the cornea, comprising a series of image frames, during the shear wave;
an image processing component that generates a plurality of difference images between adjacent image frames in the series of image frames, determines the position of the propagating shear wave in each difference image, and determines a speed of the shear wave at each of a plurality of locations from the determined position of the propagating shear wave in the plurality of difference images; and
a parameter calculation component that calculates a value for the biomechanical property from the determined speed of the shear wave at a plural subset of the plurality of locations.

17. The system of claim 16, the system further comprising a Schiempflug camera arrangement that captures video of a cross-section of tissue substantially perpendicular to a surface of the tissue, the image processing component determining a speed of the shear wave at each of the plurality of locations and another plurality of locations within the cross-section of tissue.

18. The system of claim 17, wherein the image processing component registers the determined velocities at the plurality of locations with an anatomical map of the tissue

19. The system of claim 16, wherein the biomechanical property is one of a shear modulus and Young's modulus.

20. The system of claim 16, wherein the plural subset of the plurality of locations is a proper subset.
